(19) United States
(12) Patent Application Publication
(10) Pub. No.: US 2005/0042733 A1
(43) Pub. Date: Feb. 24, 2005

(54) Method for preparing heparin from mast cell cultures

(75) Inventors: Pierre Cans, Lisses (FR); Jean-Marc Guillaume, Paris (FR); Helene Monique Marie Rigal, Morsang sur Orge (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: AVENTIS PHARMA S.A., Antony (FR)

(21) Appl. No.: 10/492,200

(22) PCT Filed: Oct. 22, 2002
(86) PCT No.: PCT/FR02/03617

(30) Foreign Application Priority Data
Oct. 22, 2001 (EP) 01/13606

(51) Int. Cl. C08B 37/10; C12P 19/26

(52) U.S. Cl. 435/84; 536/21

(57) ABSTRACT

The invention concerns the production of heparin from mast cell cultures, in particular pig mast cells.

The present invention relates to the preparation of heparin from cell cultures.

Heparin belongs to the glycosaminoglycan (GAG) family, which includes the linear polysaccharides containing a repeat of a disaccharide sequence made up of an amino sugar (D-glucosamine or galactosamine) and a uronic acid (D-glucuronic or iduronic).

In the case of heparin, which belongs, with heparan sulfate, to the glucosaminoglycan subfamily, the amino sugar is D-glucosamine. The uronic acid is either glucuronic acid (Glc) or iduronic acid (Ido). The glucosamine can be N-acetylated, N-sulfated or O-sulfated.

Conventionally, the term “heparin” refers to highly sulfated polysaccharides in which more than 80% of the glucosamine residues are N-sulfated and the number of O-sulfates is greater than that of the N-sulfates. The sulfate/disaccharide ratio is generally greater than 2 for heparin. However, the structure of heparin is in fact very heterogeneous, and chains which can contain very different ratios exist.

Like all GAGs, heparin is synthesized in the form of a proteoglycan. This synthesis takes place preferentially in a subpopulation of mast cells, serous or connective tissue mast cells (CTMCs). These mast cells are abundant in the skin and the respiratory submucosae. They have a very long lifespan (at least 6 months). Besides heparin, they contain heparan sulfate and appreciable amounts of histamine (approximately 10 pg/cell, according to the animal species).

The first step of heparin synthesis is the formation of the serglycine protein core consisting of regularly alternating serine and glycine residues. Elongation of the heparin chain takes place from a tetrasaccharide, by successive additions of osamine and of uronic acids.

The proteoglycan thus formed undergoes many sequential transformations: N-deacetylation, N-sulfation, D-glucuronic acid epimerization, and O-sulfation.

However, this complete maturation only takes place on part of the proteoglycan, which generates a great structural variability of heparin, responsible for its heterogeneity.

The polysaccharide chains are then cleaved from the serglycine by an endoglucuronidase. These chains then have a molecular weight of between 5 000 and 30 000 Da. They form complexes with alkaline proteases and are thus stored in the mast cell granules. Heparin is excreted only during mast cell degranulation.

Heparin plays an important biological role, in particular in hemostasis, and is very widely used in therapeutics, in particular as an anticoagulant and an antithrombotic agent.

Currently, most of the heparin used is isolated from pig intestinal mucosa, from where it is extracted by proteolysis, followed by purification on anion exchange resin (for a review on the various methods for preparing heparin, cf. DUCLOS; “L'Héparine: fabrication, structure, proprietes, analyse”; Ed. Masson, Paris, 1984).

Added to the inherent heterogeneity of heparin is the diversity of the batches of animals from which it is obtained. A very substantial variability results therefrom, reflected in particular in the level of biological activity. In addition, it is difficult to regularly have a sufficient supply of raw material.

The use of cells derived from mammals for producing GAG or proteoglycans has already been proposed.

Thus, application WO 99/26983 describes the obtaining of compounds of the heparin type, which may be proteoglycans (HEP-PG) or glycosaminoglycans (HEP-GAG), from rat mast cells. The compounds are not heparin. The cells thus isolated are not established lines. In addition, the applicant recommends coculturing the isolated cells with fibroblasts.

The article by Wang and Kovanen (Circulation Research, 84, 1, 74-83, 1999) itself also describes the isolation of rat serous mast cells and the production of proteoglycans from these cells. As in application WO 99/26983, the cells used for the production of proteoglycans are not established lines, but simply cells which have been isolated and then stimulated to produce proteoglycans.

Application WO 90/14418, cited in the search report, describes cell lines obtained from mouse mastocytomas and their use for the production of heparin. The origin of these cells is therefore tumoral, which may raise health problems. An article by Montgomery et al. (Proc Natl Acad Sci USA, 89, 23, 11327-11331, 1992) itself also describes the isolation of mouse mastocytomas.

The present invention proposes to overcome the drawbacks mentioned above and to avoid problems of supply in terms of quantity and of quality, using a conveniently available source of homogeneous raw material, with stable characteristics, facilitating the production of preparations of heparin of constant quality.

The inventors have noted that it is possible to produce, from mast cell line cultures, a considerable amount of heparin having properties comparable to those of the heparin extracted from pig intestinal mucosa. The use of cell cultures as raw material also makes it possible to control the conditions for synthesizing the heparin, and to thus obtain a product having reproducible characteristics.

A subject of the present invention is a method for producing heparin, characterized in that it comprises culturing mast cells of porcine origin and recovering the heparin from the cultures obtained.

Preferably, said mast cell cultures are mast cell lines of porcine origin.

The term “culture” here denotes, in general, a cell or a set of cells grown in vitro. A culture developed directly from a cell or tissue sample taken from an animal is referred to as a “primary culture”. The term “line” is employed when at least one passage, and generally several consecutive passages in subculture, have been successfully performed, and denotes any culture which is derived therefrom (SCHAEFFER, In Vitro Cellular and Developmental Biology, 26, 91-101, 1990).

Advantageously, said mast cells are derived from porcine mast cell cultures and in particular from porcine mast cell lines obtained as described in Application FR 0113608, and also in the PCT application entitled “Cultures de mastocytes de porc et leurs utilisations” [pig mast cell cultures and their uses] in the name of INRA and of ENVA filed on the same day as the present application. Among these, preferred lines for implementing the method in accordance with the invention are:
the mast cell line derived from pig fetal liver deposited by INRA (147 rue de l'Université, 75007 Paris, France) with the CNCM (Collection Nationale de Cultures de Microorganismes [National Collection of Cultures of Microorganisms], Pasteur Institute, 26, rue du Docteur Roux, 75724 PARIS CEDEX 15, France) on Oct. 17, 2001, under the number I-2735;
the mast cell line derived from pig fetal liver and transfected with the SV40 virus T antigen, deposited by INRA with the CNCM on Oct. 17, 2001, under the number I-2736;
the mast cell line derived from pig fetal bone marrow and transfected with the SV40 virus T antigen, deposited by INRA with the CNCM on Oct. 17, 2001, under the number I-2734.

Preferably, these mast cells are serous mast cells.

These mast cells will preferably be cultured in a defined culture medium (MEMα/DMEM, RPMI, IMDM, etc.) supplemented with growth factors, used in combination or individually, such as SCF (Stem Cell Factor) at a concentration of between 1 ng/ml and 1 μg/ml and, optionally, IL3 (interleukin 3) at a concentration of between 0.1 ng/ml and 100 ng/ml, or PGE2 (prostaglandin E2) at a concentration of between 1 nM and 1 μM.

The media may also be supplemented with bovine serum, at a concentration of between 0.5% and 20% (v/v).

The addition of bovine serum to the culture media can be replaced with the use of a serum-free culture medium such as AIMV (INVITROGEN) so as to reduce the protein concentration of the medium and the risks associated with the use of compounds of animal origin (KAMBE et al., J. Immunol. Methods, 240, 101-10, 200).

It is possible to obtain cells which do not depend on the addition of serum and/or the use of growth factors by controlled mutation of the cell phenotype through the action of transformer and/or immortalizing agents (TSUJIMURA, Pathology International, 46, 933-8, 1996; PIAO and BERNSTEIN, Blood, 87(8), 3117-23, 1996).

The mast cells can be cultured using the techniques developed for the mass culture of eukaryotic cells, as described, for example, by GRIFFITHS et al. (Animal Cell Biology, Eds. Spier and Griffiths, Academic Press, London, Vol. 3, 179-220, 1986). It is possible to use bioreactors with a volume greater than several m³, as described by PHILIPS et al. (Large Scale Mammalian Cell Culture, Eds. Feder and Tolbert, Academic Press, Orlando, USA, 1985) or by MIZRAHI (Process Biochem, Aug. 9-12, 1983).

The culturing can also be carried out in suspension or on a microsupport according to the technique described by VAN MEZEL (Nature, 216, 64-65, 1967).

It is also possible to use batch culturing systems, which are commonly used for eukaryotic cell cultures due to the fact that they are much simpler to use on an industrial scale (VOGEL and TODARO, Fermentation and Biochemical Engineering Handbook, 2^{nd} edition, Noyes Publication, Westwood, N.J., USA, 1997). The cell densities obtained with these systems are generally between 10⁶ and 5×10⁶ cells/ml.

The productivity of the batch cultures can advantageously be increased by removing some of the cells from the bioreactor (70% to 90%) for the GAG extraction and heparin isolation operations, and keeping the remaining cells within the same bioreactor in order to initiate a new culture. In this “repeated batch” culturing mode, it is also possible to distinguish the optimum parameters of the cell growth phase from those which allow greater accumulation of GAGs and of heparin within the cells.

Continuous perfusion-fed culture systems, with or without cell retention, can also be used (VELEZ at al., J. Immunol. Methods, 102(2), 275-278, 1987; CHAUBARD et al., Gen. Eng. News, 20, 18-48, 2000). In the context of the present invention, use may in particular be made of perfusion-fed culture systems which allow cells to be retained within the reactor, and which result in a growth and a production greater than those which can be obtained in batch culture. The retention can be effected by virtue of retention systems of the spin-filter, hollow fiber or solid matrix type (WANG et al., Cytotechnology, 9, 41-49, 1992; VELEZ et al., J. Immunol. Methods, 102(2), 275-278, 1987). The cell densities obtained are generally between 10⁷ and 5×10⁷ cells/ml. Culturing in bioreactors allows, through the use of on-line measuring sensors, better control of the physicochemical parameters of the cell growth and also of the accumulation of GAGs and of heparin within the cells: pH, PO₂, Red/Ox, growth substrates such as vitamins, amino acids, carbon-based substrates (for example glucose, fructose, galactose), metabolites such as lactate or aqueous ammonia, etc.

The cells can be harvested and separated from the culture medium, generally by centrifugation or filtration, after from 3 to 30 days of culturing, generally after from 3 to 10 days of culturing, under these conditions.

Various centrifugation systems can be used; mention will, for example, be made of those described by VOGEL and TODARO (Fermentation and Biochemical Engineering Handbook, 2^{nd} Edition, Noyes Publication, Westwood, N.J., USA).

Alternatively, or in combination with centrifugation, the separation may be carried out by tangential microfiltration using membranes the porosity of which is less than the average diameter of the cells (5 to 20 μm) while at the same time allowing the other compounds in solution/suspension to pass through. The rate of tangential flow and the pressure applied to the membrane will be chosen so as to generate little shear force (Reynolds number less than 5 000 sec⁻¹) in order to reduce clogging of the membranes and to preserve the integrity of the cells during the separating operation.

Various membranes can be used, for example spiral membranes (AMICON, MILLIPORE), flat membranes or hollow fibers (AMICON, MILLIPORE, SARTORIUS, PALL, GF).

It is also possible to choose membranes the porosity, the charge or the grafting of which makes it possible to perform a separation and a first purification with respect to possible contaminants which may be present in the culture medium, such as cell proteins, DNA, viruses, or other macromolecules.

Use may be made of methods of production and of cell harvesting which make it possible to conserve the GAGs and the heparin in the intracellular content; however, the GAGs and the heparin can also be harvested from the culture medium after lysis or degranulation of the cells.

The degranulation may be caused by the binding of specific ligands to the receptors present at the surface of the mast cells, for example the binding of allergen-type agents (such as IgE Fc fragment or analogs of this fragment) to the mast cell IgE receptors. When the heparin has been released from the intracellular content, by degranulation or lysis of all or some of the mast cells, and is present in the culture medium at the time of the separation step, the use of membranes with a smaller porosity may also be envisaged. In this case, the cell separation is combined with a step consisting of ultrafiltration on one or more membranes, the organization and the porosity of which make it possible to concentrate the heparin and to separate it from the other species present in the medium, as a function of the size and the molecular weight and, optionally, of the electrical charge, or of the biological properties.

In the context of this embodiment, the cutoff threshold of the membranes is preferably between 1000 and 5 kDa. Use may be made of membrane systems similar to those used for microfiltration, for example spiral membranes, flat membranes or hollow fibers. Use may advantageously be made of membranes which make it possible to separate and purify the heparin due to their charge properties or their properties of grafting of ligands exhibiting affinity for heparin (for example antibodies, ATIII, lectin, peptides, nucleotides, etc.).

Other agents can also induce mast-cell degranulation. These agents can be classified in several categories, such as cytotoxic agents, enzymes, polysaccharides, lectins, anaphylatoxins, basic compounds (compound 48/80, substance P, etc.), calcium (A23187 ionophore, ionomycin, etc.) [D. Lagunoff and T. W. Martin, 1983, Agents that release histamine from mast cells. Ann. Rev. Pharmacol. Toxicol., 23:331-51]. A degranulating agent can be used repeatedly on the same cells maintained in culture. In this method of production, the productivity is increased significantly by the simplification of the method of harvesting from the supernatant and by the maintaining of the cells in culture.

In the particular case of A23187 ionophore, the mast-cell degranulation can be induced, for example, by treatment of 2×10⁶ mast cells/ml with the A23187 ionophore at concentrations between 1 and 100 μg/ml and action times ranging from 1 minute to 4 hours.

The mast-cell lysis can be induced, for example, by osmotic shock using hypotonic or hypertonic solutions, by thermal shock (freezing/thawing), by mechanical shock (for example sonication or pressure variation), by the action of chemical agents (NaOH, THESIT™, NP40™, TWEEN 20™, BRIJ-58™, TRITON X™-100, etc.) or by enzyme lysis (papain, trypsin, etc.), or by a combination of two or more of these methods.

To extract and purify the heparin from the cell lysate, to separate the polysaccharide chains from the serglycine core, and to separate the heparin chains from the other GAGs present in the extraction medium, use may be made of methods similar to those used in the context of the extraction and purification of heparin from animal tissues, which are known in themselves, and described in general works such as the manual by DUCLOS (mentioned above).

By way of nonlimiting examples, in order to separate the heparin from the nucleic acids and from the cell proteins, and to solubilize it, i.e. to break the bonds with the serglycine core:
the cell lysate can be subjected to one or more enzyme digestions (pronase, trypsin, papain, etc.);
the heparin-protein bonds can be hydrolyzed in alkali medium, in the presence of sulfates or chlorides;
it is also possible to carry out a treatment in acid medium (for example with trichloroacetic acid under cold conditions) in order to destroy the nucleic acids and the proteins originating from the cells, to which is added the use of an ionic solution which makes it possible to dissociate the GAG-protein interactions;
it is also possible to carry out an extraction with guanidine, after enzyme hydrolysis; to purify the solubilized heparin, it is possible, for example, to precipitate it with potassium acetate, with a quaternary ammonium, with acetone, etc.

These purification steps can advantageously have added to them or be replaced with one or more chromatography steps, in particular anion exchange chromatography or affinity chromatography steps.

A subject of the present invention is also the heparin preparations which can be obtained from mast cell cultures using a method according to the invention.

The heparin preparations in accordance with the invention, which have biological properties comparable to those of the heparin preparations obtained in the prior art from animal tissues, can be used in all the usual applications for heparin.

The present invention will be understood more clearly from the additional description which follows, which refers to examples of preparing heparin from mast cell cultures and of characterizing the heparin obtained.

### EXAMPLE 1

### Extraction of Heparin from Mast Cell Cultures

Culturing of Mast Cells

A pig fetal liver mast cell line and a line of pig fetal liver mast cells transfected with the SV40 virus T antigen (lines CNCM I-2735 and CNCM I-2736, respectively) were used.

The cells are seeded at a rate of 10⁵ to 5×10⁵ cells/ml, in complete MEMα medium in the presence of porcine IL3 (2 ng/ml) and of porcine SCF (80 ng/ml).

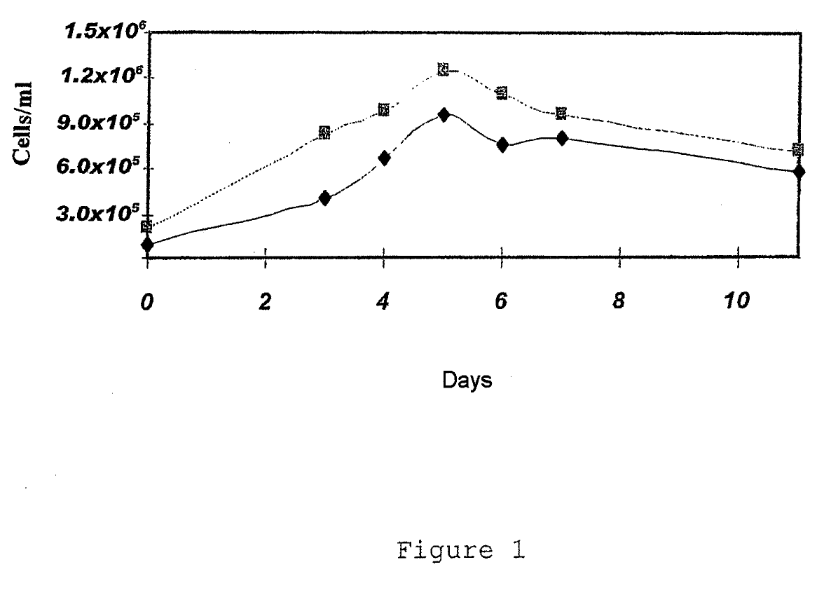

The cultures are prepared in a culture dish or in suspension in a 1-liter spinner flask. The cell growth is monitored daily for 4 to 12 days. The heparin production is monitored in parallel, by analyzing the glycosaminoglycans produced in culture. The results are given in FIGS. 1 to 5.

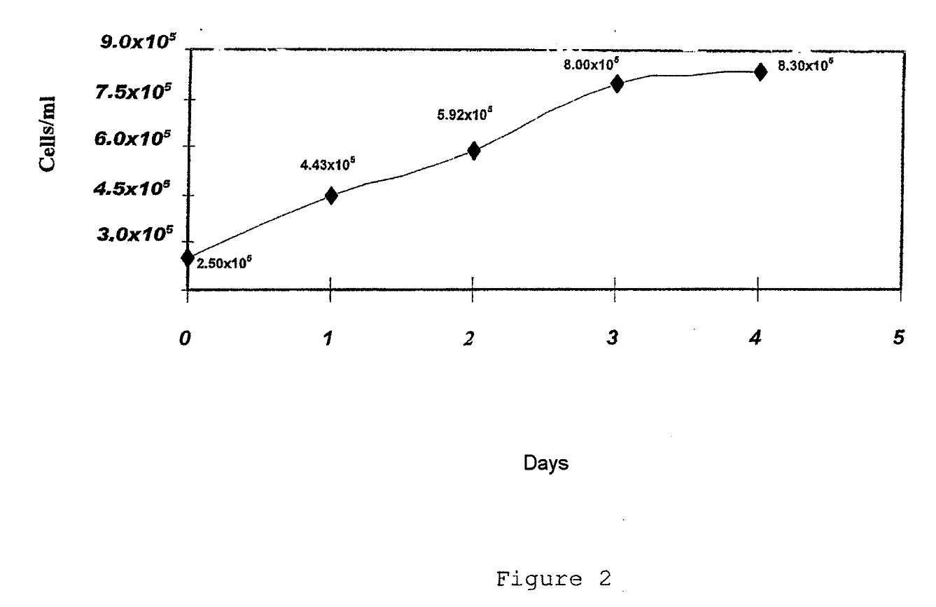

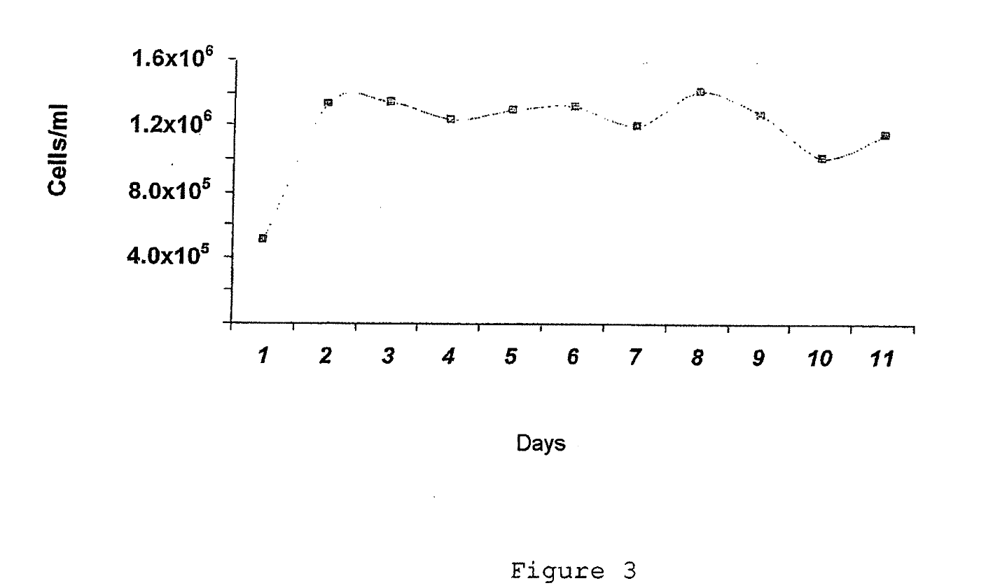

FIGS. 1, 2 and 3 illustrate the growth of liver mast cells in static culture in dishes (FIG. 1; initial seeding: ♦: 1×10⁵ cells; ▪: 2×10⁵ cells) and in suspension in flasks (FIG. 2), and the growth of transfected liver mast cells in suspension in flasks (FIG. 3).

In these experiments, the cultures in suspension in flasks exhibit a maximum cell density ranging from approximately 8×10⁵ (for the nontransfected cells) to approximately 1.5×10⁶ cells/ml (for the transfected cells). The doubling time, calculated during the exponential growth phase, is between 24 and 48 hours.

Glycosaminoglycan Purification

The cells undergo hydrolysis in alkali medium in the presence of salt in order to cleave the proteoglycans and avoid ionic GAG/protein interactions.

This treatment comprises the following steps:

1. Treatment with sodium hydroxide in saline medium: this step is aimed at destroying the cells and at cleaving the bonds between the heparin and its mother protein.

The step comprises the addition of 100 μl of 1 M NaOH and of 800 μl of 0.5 M NaCl to a pellet of 10⁶ cells. The mixture thus obtained is heated in a water bath at 80° C. for 30 minutes, and then sonicated for 5 minutes before being neutralized with 1 N HCL.

2. Extraction: the hydrolyzed sample is loaded onto an anion exchange resin column (SAX, Varian), which retains heparin. The column is washed three times in Tris/HCl buffer, pH 7.4, containing 0.5 M NaCl in order to eliminate the proteins and the other GAGs, in particular the dermatan. The heparin is then eluted with 1 ml of Tris/HCl buffer, pH 7.4, containing 3 M NaCl.

3. Desalting/lyophilization: the elimination of the sodium chloride (necessary in order to be able to apply some of the analytical methods which are described below) is carried out by steric exclusion chromatography on SEPHADEX G10 gel, followed by conductimetry. The collected heparin fractions are then lyophilized so as to concentrate the sample.

Analysis by Polyacrylamide Gel Electrophoresis

This technique makes it possible to separate the GAGs according to their size and their charge, and constitutes a test for rapidly verifying the presence or absence of heparin.

The purified preparation obtained as described above is loaded onto a Tris/tricine polyacrylamide gel (gradient from 10 to 20%) for separating molecules of 30 to 1 kDa, in a proportion of 20 μl of preparation per deposit. 25 ng of dermatan, and 25 ng of SPIM standard porcine heparin (4^{th} international standard for porcine heparin from intestinal mucosa), and of the heparin extracted from porcine mucosa and purified by treatment with sodium hydroxide and purification on anion exchange resin under the same conditions as those described above are loaded onto the same gel.

Double staining with a solution of alcian blue and then silver nitrate as described in AL-HAKIM and LINHARDT (Applied and Theoretical Electrophoresis 1, 305-12, 1991) makes it possible to reveal the glycosaminoglycans (silver nitrate alone only reveals proteins).

The gels are then analyzed with a scanner (BIO-RAD) in order to quantify the various GAGs. The heparin quantification limit is 10 ng per band.

The results of an experiment are summarized in Table 1 below, in which the amount of heparin produced by the cells is expressed as μg/10⁶ cells.

TABLE 1

| | Days of harvesting | | | | | | | |
|---|---|---|---|---|---|---|---|---|
| | 3 | 4 | 5 | 6 | 7 | 10 | 11 | 14 |
| Liver cells, dish | 2.6 | 3.5 | 4.4 | 6.5 | 3.7 | 4.2 | — | 8.1 |
| Transfected liver | 2.6 | 6.9 | 9.0 | 11.7 | 10.8 | 8.5 | 5.4 | 7.1 |
| cells, dish | | | | | | | | |
| Liver cells, flask | 1.2 | — | — | — | — | — | — | — |
| Transfected liver | — | 2.1 | — | — | — | — | — | — |
| cells, flask | | | | | | | | |

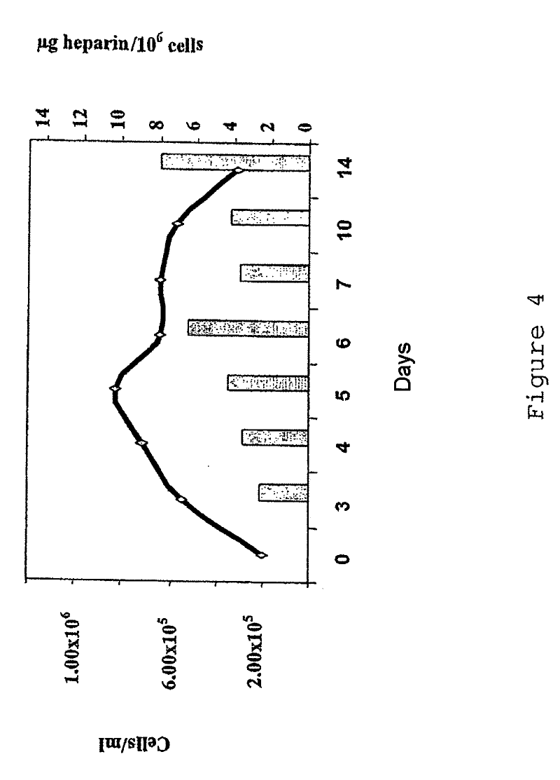

These results are also illustrated in FIG. 4 (curve=cell population; bars=heparin production).

FIG. 4 illustrates the heparin production during growth of the liver mast cells in static culture in dishes.

The heparin concentrations generally observed are between 2 and 14 μg per 10⁶ cells, in static culture or in suspension.

### EXAMPLE 2

### Characterization of the Preparation of Heparin Obtained from Mast Cell Cultures

Disaccharide Profile by HPLC

The disaccharide composition makes it possible to differentiate the heparin from the other glycosaminoglycans.

The disaccharide profile of the glycosaminoglycans produced by the mast cells in culture was determined according to the method described by LINHARDT et al. (Biomethods, 9, 183-97, 1997).

The GAG preparation obtained as described in Example 1 above was depolymerized with a mixture of Flavo-bacterium heparinium heparinases (heparinases I, II and III, GRAMPIAN ENZYMES). The conditions used are described in the publication by LINHARDT et al., mentioned above.

As a control, the SPIM standard heparin was depolymerized under the same conditions.

Under these conditions, the depolymerization is complete and produces disaccharides.

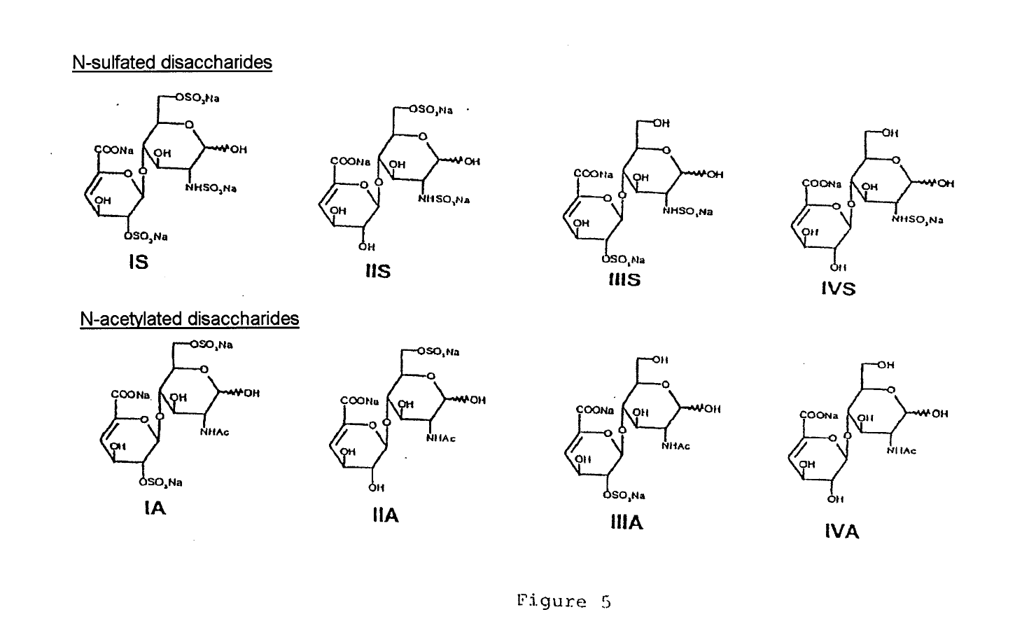

The main disaccharides, eight in number, which are either N-sulfated or N-acetylated, are represented in FIG. 5.

UV Detection

These disaccharides are separated and identified by HPLC, on an anion exchange column as described by LINHARDT et al. (mentioned above).

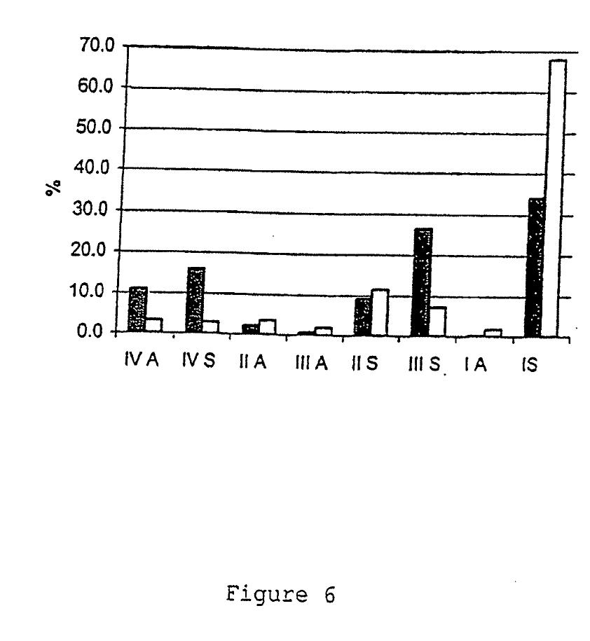

The results are illustrated in FIG. 6, representing the disaccharide profile of the preparation of heparin produced by a flask culture of fetal liver-derived mast cells (▪), compared to the disaccharide profile of the standard heparin (□).

These results show that all the disaccharides present in the SPIM reference porcine heparin are also present in the mast cell heparin, although in different proportions. The IS/IIS ratio is 3.7.

Fluorescence Detection

A similar method with fluorimetric detection makes it possible to quantify only the IS and IIS disaccharides, characteristic of heparin, and to calculate the ratio thereof.

The enzymatic depolymerization and the HPLC separation are carried out in the same way as that described above.

The separation is followed by a post-column derivatization, so as to form a fluorescent complex with guanidine.

The IS trisulfated disaccharide, which has the strongest response factor by this technique, is detected and quantified with respect to a solution of standard heparin of known concentration.

The detection limit of the method is of the order of 5 ng/ml of heparin in the cell culture samples.

Table 2 below illustrates the IS/IIS ratio of cell cultures over time.

TABLE 2

| | Days of harvesting | | | | | | | |
|---|---|---|---|---|---|---|---|---|
| | 3 | 4 | 5 | 6 | 7 | 10 | 11 | 14 |
| Liver cells, dish | 1.9 | 1.6 | 1.6 | 1.4 | 1.4 | 1.3 | — | 1.4 |
| Transfected liver | 4.1 | 5 | 4.6 | 6.6 | 3.7 | 4.9 | 5.6 | 5.7 |
| cells, dish | | | | | | | | |
| Liver cells, flask | 2.3 | — | — | — | — | — | — | — |
| Transfected liver | — | 2.9 | — | — | — | — | — | — |
| cells, flask | | | | | | | | |

### EXAMPLE 3

### Biological Characterization of the Heparin by Determination of the Anti-Xa and Anti-IIa Activities

Biological Activities

Inactivation of factors Xa and IIa is characteristic of heparin, and makes it possible to differentiate it from heparan sulfate and from dermatan.

The method used is that described in the European Pharmacopoeia, 3^{rd} edition (1997), monograph on low molecular weight heparins.

The reaction occurs in three steps:
1. ATIII+heparin→[ATIII−heparin]
2. [ATIII−heparin]+factor(excess)→([ATIII−heparin−factor]+factor(residual)
3. factor(residual)+chromophore substrate→pNA

The amount of para-nitroaniline (pNA) released is measured at 405 nm. It is inversely proportional to the amount of heparin.

The anti-Xa or anti-IIa activity is evaluated with respect to a calibration straight line established with the SPIM standard.

The sensitivity of the method is 0.006 IU/ml.

The results obtained are given in Table 3 below.

TABLE 3

| | Days of harvesting | | | | | | | |
|---|---|---|---|---|---|---|---|---|
| | 3 | 4 | 5 | 6 | 7 | 10 | 11 | 14 |
| Liver cells, | | | | | | | | |
| dish: | | | | | | | | |
| Anti-Xa | 2.1 | 1.8 | 5.7 | 4.0 | 2.4 | 2.2 | — | 0.0 |
| Anti-IIa | — | — | — | — | — | — | — | — |
| Anti-Xa/anti- | — | — | — | — | — | — | — | — |
| IIa ratio | | | | | | | | |
| Transfected | | | | | | | | |
| liver cells, | | | | | | | | |
| dish: | | | | | | | | |
| Anti-Xa | 44 | 11.5 | 11.7 | 12.3 | 11.4 | 13.0 | 11.6 | 12.9 |
| Anti-IIa | — | — | — | — | — | — | — | — |
| Anti-Xa/anti- | — | — | — | — | — | — | — | — |
| IIa ratio | | | | | | | | |
| Liver cells, | | | | | | | | |
| flask: | | | | | | | | |
| Anti-Xa | 0.7 | — | — | — | — | — | — | — |
| Anti-IIa | 1.4 | — | — | — | — | — | — | — |
| Anti-Xa/anti- | 0.6 | — | — | — | — | — | — | — |
| IIa ratio | | | | | | | | |
| Transfected | | | | | | | | |
| liver | | | | | | | | |
| cells, flask: | | | | | | | | |
| Anti-Xa | — | 3.1 | — | — | — | — | — | — |
| Anti-IIa | — | 14 | — | — | — | — | — | — |
| Anti-Xa/anti- | — | 0.2 | — | — | — | — | — | — |
| IIa ratio | | | | | | | | |

The anti-Xa or anti-IIa activity of the heparin obtained from mast cells in culture was compared with the anti-Xa or anti-IIa activity, respectively, of the heparin obtained from porcine mucosa or of the standard heparin. The results are illustrated in Table 4 below.

TABLE 4

| | Anti-Xa | Anti-IIa | Xa/IIa |
|---|---|---|---|
| | (IU/mg) | (IU/mg) | (IU/mg) |
| Mast cell heparin | 18 to 3.1 | 14 to 3 | 0.2 to 1 |
| Mucosal heparin | 80 | 81 | 1 |
| Standard heparin | 180 | 180 | 1 |

Characterization of the ATIII Binding

The binding between heparin and ATIII is demonstrated by a migration shift using electrophoresis techniques as described in LEE and LANDER (Proc. Natl. Acad. Sci., 88, 2768-72, 1991).

The electrophoresis is carried out on a 0.8% agarose gel in a solution of pH 3 (acetic acid/lithium hydroxide).

100 μl of ATIII (human origin; BIOGENIC) solution at decreasing concentrations of 584 to 183 μg/ml are added to 100 μl of test sample.

100-μl deposits of sample are loaded. The migration is for 30 minutes at 100 volts.

The gels are fixed with a solution of 0.1% hexadecyltrimethylammonium bromide (CETAVLON-SIGMA).

Revelation is carried out with Azure A (0.08% in water).

The gels are scanned and interpreted with the QUANTITY ONE software (BIO-RAD).

The results are expressed as % heparin bound to ATIII.

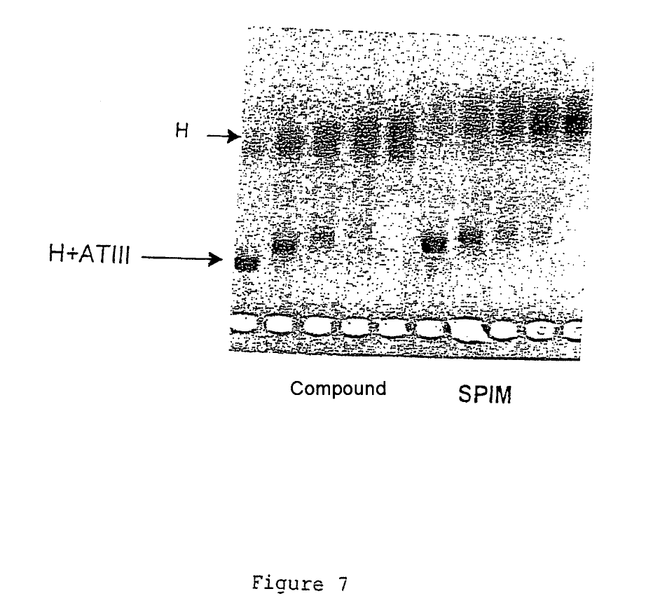

The results obtained in the case of a flask culture of transfected liver cells are illustrated in FIG. 7.

31% ATIII binding (theoretical value 33%) is observed in the presence of standard heparin (SPIM), and 27% ATIII binding in the presence of the heparin obtained from mast cells in culture (compound).

### EXAMPLE 4

### Culturing of Mast Cells in a Repeated Batch Bioreactor

An untransfected line of mast cells derived from porcine fetal liver was used. The cells are seeded at a rate of 2.0 to 4.0×10⁵ cells per ml in complete DMEM/F12 medium supplemented with porcine IL3 (2 ng/ml) and porcine SCF (80 ng/ml).

The bioreactor used has a volume of 2 liters of culture medium, the oxygen tension of the culture is maintained at between 20% and 40% of saturation, the pH is maintained between 7.0 and 7.4, and the temperature is maintained at 37° C.+/−0.5° C. by circulation of thermostated water in the bioreactor jacket. The culture is stirred using a marine propeller, with a rate of between 80 and 150 rpm.

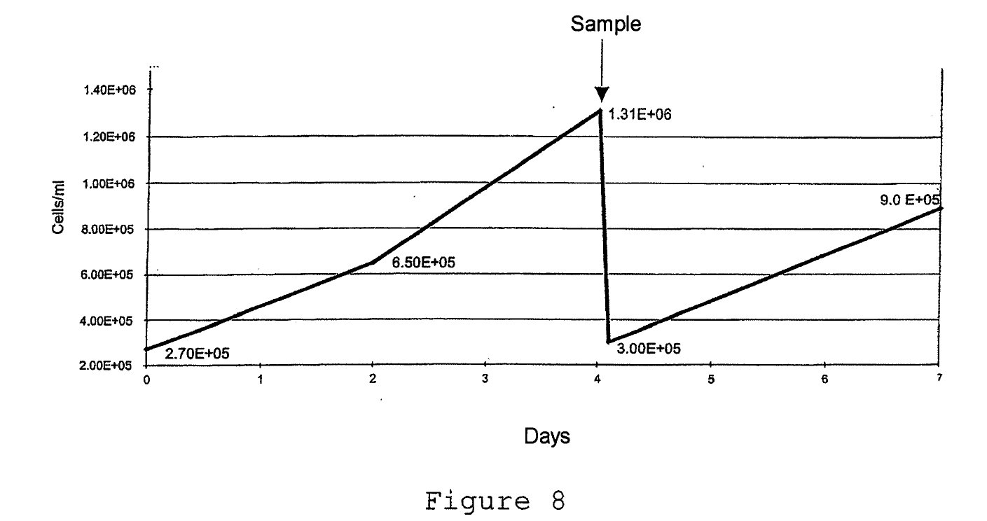

After culturing for 4 days, the cell density is 1.3×10⁶ cells/ml, corresponding to a doubling time of between 24 and 48 h. On the day of harvesting, 80% of the culture is removed for the heparin extraction, and the remainder of the culture is kept in the bioreactor and diluted with fresh medium to a concentration of between 2.0 and 3.0×10⁵ cells/ml as described for a repeated-batch production operation. Three days after dilution in repeated-batch mode, the cell density obtained is 9.0×10⁵ cells/ml, corresponding to a doubling time of between 24 and 48 hours and comparable to the first culturing (FIG. 8).

The heparin is purified as described in Example 1.

Purified heparin is then analyzed by HPLC, as described in Example 2, using the SPIM standard heparin as control.

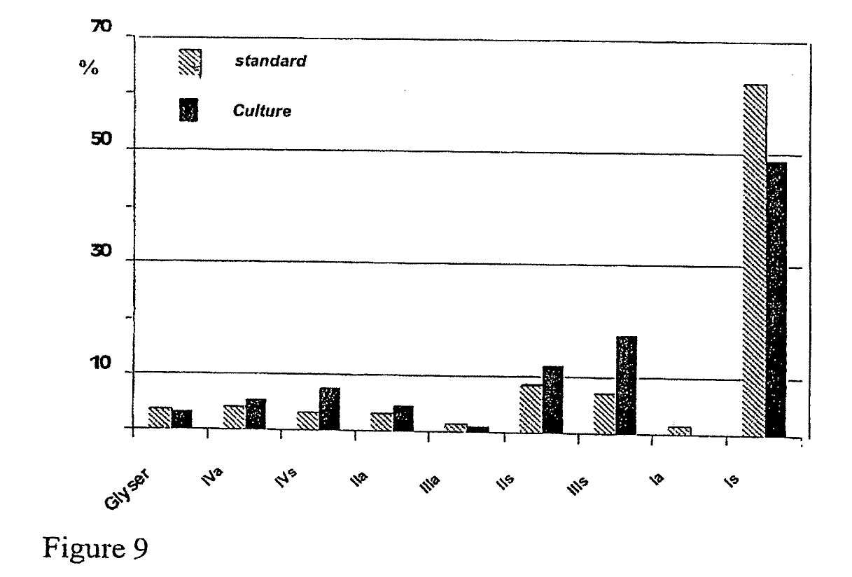

Table 5 and FIG. 9 represent the disaccharide profile and the proportion of the serglycine (Gly-Ser) protein core of the preparation of heparin produced by suspension-culturing of mast cells derived from porcine fetal liver (▪), compared to the profile obtained for the SPIM standard heparin (□).

Table 6 represents the N-acetylation, N-sulfation and O-sulfation profile of the disaccharides of the heparin produced by suspension-culturing of mast cells derived from porcine fetal liver, compared to that of the disaccharides of the standard SPIM heparin.

TABLE 5

| | % Standard | % Culture |
|---|---|---|
| Gly-Ser | 3.5 | 3.2 |
| IVa | 4 | 5.4 |
| IVs | 3.1 | 7.6 |
| IIa | 3.1 | 4.4 |
| IIIa | 1.5 | 0.7 |
| IIs | 8.4 | 11.9 |
| IIIs | 7.2 | 17.1 |
| Ia | 1.3 | 0.2 |
| Is | 62 | 48.8 |

TABLE 6

| Disaccharides | % Standard | % Culture |
|---|---|---|
| Acetylated | 11.8 | 10.7 |
| 2-O-sulfated | 23 | 8 |
| 6-O-sulfated | 42 | 42 |
| N-sulfated | 83 | 85 |
| 2-O-sulfated | 84 | 77 |
| 6-O-sulfated | 89 | 71 |
| Sulfates/carboxylates | 2.4 | 2.1 |

Similar results are obtained when a line of mast cells transfected with the SV40 virus T antigen is used.

### EXAMPLE 5

### Production of Heparin in the Culture Supernatant Using a Degranulating Agent

The experiments were carried out on a line of untransfected fetal liver mast cells.

On the 762^{nd} day (counting from the first culturing) the mast cell concentration was adjusted to 2×10⁶ cells/ml, and the culture was incubated for one hour in MEM medium comprising 4 μg/ml of the ionophore A23187, which induces mast cell degranulation.

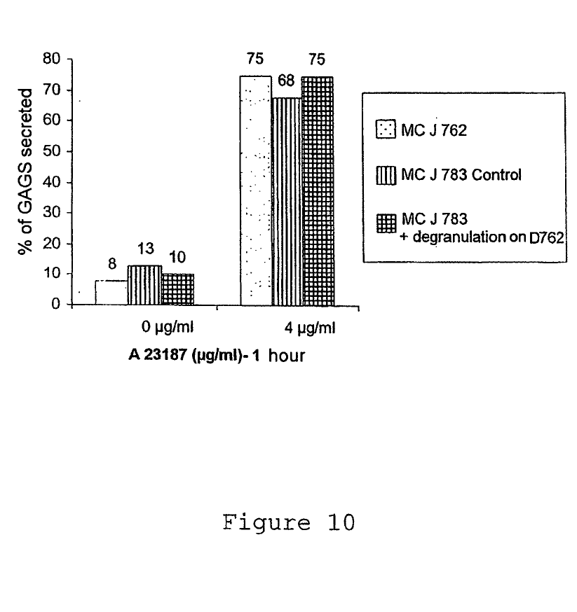

The total GAGs and the secreted GAGs produced by the cells are quantified by PAGE. FIG. 10 shows that 70 to 75% of the GAGs are found in the supernatant after treatment with the ionophore A23187, versus approximately 10% in the nontreated cells (0 μg/ml of A23187).

The mast cells for which the GAG harvesting was carried out on the 762^{nd} day of culturing were placed in culture again. No loss of viability or of growth rate was observed.

21 days later, these mast cells were subjected to a further degranulation, and the GAGs were assayed as described above. A mast cell culture of the same age, which had not undergone degranulation on the 762^{nd} day was used as a control.

The results are given in FIG. 10, which shows that the percentage of GAGs secreted is comparable with that obtained during the first degranulation and also comparable to that obtained with the control cells of the same age.

Similar results are obtained when a line of mast cells transfected with the SV40 virus T antigen is used.

1. A method for producing heparin, which comprises:
culturing mast cells of porcine origin and
recovering the heparin from the cultures obtained.

2. The method as claimed in claim 1, wherein said mast cell cultures are mast cells lines of porcine origin.

3. The method as claimed in claim 1, wherein said mast cells are derived from pig fetal bone marrow or pig fetal liver.

4. The method as claimed in claim 1, said mast cells are serous mast cells.

5. The method as claimed in claim 1, wherein said mast cells are derived from a mast cell line selected from the group consisting of
the line deposited with the CNCM [National Collection of Cultures of Microorganisms] on Oct. 17, 2001, under the number I 2735;
the line deposited with the CNCM on Oct. 17, 2001, under the number I 2736; and
the line deposited with the CNCM on Oct. 17, 2001, under the number I 2734.

6. A preparation of heparin which is prepared by the process as claimed in claim 1.
